(19) United States
(12) Patent Application Publication
(10) Pub. No.: US 2015/0129457 A1
(43) Pub. Date: May 14, 2015

(54) PHARMACEUTICAL COMPOSITIONS FOR INTRAOCULAR ADMINISTRATION AND METHODS FOR FABRICATING THEREOF

(71) Applicant: IMPRIMIS PHARMACEUTICALS, INC., San Diego, CA (US)

(72) Inventors: Forest J. Flodin, San Diego, CA (US); John P. Saharek, San Diego, CA (US); Mark L. Baum, San Diego, CA (US); Richard Dilzer, Long Valley, NJ (US)

(73) Assignee: IMPRIMIS PHARMACEUTICALS, INC., San Diego, CA (US)

(21) Appl. No.: 14/596,865

(22) Filed: Jan. 14, 2015

Related U.S. Application Data
(63) Continuation-in-part of application No. 14/227,819, filed on Mar. 27, 2014.
(60) Provisional application No. 61/958,170, filed on Jul. 22, 2013.

(51) Int. Cl. A61F 9/00; A61K 31/58; A61J 7/00; A61F 17/00; A61B 19/02; A61K 9/00; A61K 31/4709

(52) U.S. Cl. CPC A61F 9/0008; A61K 9/0048; A61K 31/58; A61K 31/4709; A61F 17/00; A61B 19/02; A61J 7/0015
USPC 206/572; 206/232; 206/370; 206/438

(57) ABSTRACT

Pharmaceutical compositions for intraocular administration are described, the compositions consisting essentially of a therapeutically effective quantity of an anti-bacterial agent (such as moxifloxacin), a therapeutically effective quantity of an anti-inflammatory agent (such as triamcinolone), at least one pharmaceutically acceptable excipient and a pharmaceutically acceptable carrier. Methods for fabricating the compositions and using them for intraocular administration are also described as well as pharmaceutical kits designed for administering the compositions.

### CROSS REFERENCE TO RELATED APPLICATIONS

This is a continuation-in-part patent application of U.S. patent application Ser. No. 14/227,819 filed on Mar. 27, 2014 entitled “Pharmaceutical Compositions for Intraocular Administration and Methods for Fabricating Thereof,” and claims priority under 35 U.S.C. §120 to the same, which in turn claims priority under 35 U.S.C. §119(e) to U.S. Provisional Application No. 61/958,170 filed on Jul. 22, 2013 entitled “Pharmaceutical Compositions for Intraocular Administration and Methods for Fabricating Thereof,” the entire contents of each of which is hereby incorporated by reference.

### FIELD OF THE INVENTION

The present invention relates generally to the field of ophthalmology and more specifically to ophthalmological compositions having anti-bacterial and anti-inflammatory properties, and to methods of preparing such compositions.

### BACKGROUND

In ophthalmological treatments and procedures, e.g., cataract surgery, pre- and post-operative eye drops are frequently used by the patients to eliminate or alleviate negative post-surgery complications such as infections, inflammation, and tissue edema. It has been reported that as many as 8% of all ocular surgery patients may suffer from infections, including the potentially catastrophic endophthalmitis, and various negative sight threatening side effects after surgery, such as inflammatory uveitis, corneal edema, and cystoid macular edema. Typically, the topical postoperative medications are prescribed for at-home use starting before and then after cataract surgery, and are typically self-administered, unless requiring a caregiver or family assistance.

These ophthalmic medication drops include anti-inflammatory and antibiotic agents and are highly effective, but require strict adherence to the treatment regimens, which is often difficult for many patients (with physical limitations or aversions to eyelid touching and manipulation) and is frequently expensive (well over $200 per procedure), causing patients' dissatisfaction. It is desirable to have an alternative procedure that would permit avoiding the necessity of the use of such post-surgery medications to save the associated post-operative trouble and expenses.

One such alternative procedure includes the intraoperative intravitreal injection by an atraumatic transzonular route that can achieve patient outcomes that are as good as, or better than, the current at-home eye drop regimen, removing the issues of compliance and medication administration accuracy. This patent specification discloses pharmaceutical compositions suitable for intraoperative ocular administration that can achieve such positive patient outcomes, and methods of fabricating and administering the same.

### BRIEF DESCRIPTION OF FIGURES

FIGS. 1 and 2 show schematically a combination storage/administration drug delivery device (the assembled and disassembled view, respectively) that can be used in kits according to some embodiments of the present invention.

FIG. 3 shows schematically another drug delivery device (comprising a squeezable vial) that can be used in kits according to some embodiments of the present invention.

FIG. 4 shows schematically how the device of FIG. 3 may be used.

FIGS. 5-7 show schematically convertible delivery devices that can be used in kits according to some embodiments of the present invention.

### SUMMARY

According to one embodiment of the invention, a pharmaceutical composition for intraocular injection is provided, the composition comprising a therapeutic component consisting essentially of a therapeutically effective quantity of an anti-bacterial agent and a therapeutically effective quantity of an anti-inflammatory agent, and at least one pharmaceutically acceptable excipient and/or a pharmaceutically acceptable carrier that are suitable for intraocular injection.

According to another embodiment of the invention, an anti-bacterial agent described herein can be a compound selected from the group of a quinolone (including a fluorinated quinolone), e.g., moxifloxacin, and a pharmaceutically acceptable salt, hydrate, solvate or N-oxide thereof.

According to yet another embodiment of the invention, an anti-inflammatory agent agent described herein can be a corticosteroid, e.g., triamcinolone, and a pharmaceutically acceptable salt, hydrate, solvate, ether, ester, acetal and ketal thereof.

According to another embodiment of the invention, the pharmaceutical compositions described herein may further include a solubilizing and suspending agent such as non-ionic polyoxyethylene-polyoxypropylene block copolymer, e.g., Poloxamer 407®.

According to other embodiments of the invention, the pharmaceutical compositions described herein may be intravitreally transzonularly injected into a mammalian subject as a part of the process of treatment of a variety of ophthalmological diseases, conditions or pathologies associated with intraocular surgery, such as cataracts, retinal and glaucoma disease.

In another embodiment of the invention, the pharmaceutical composition is within a pharmaceutical kit comprising a sealed container containing the composition, one or several drug delivery devices for administering the composition and instructions for use. In some embodiments, the sealed container may be used in conjunction with one or more delivery devices, such as an eyedropper; an injector, such as a needle or cannula; a snap eyedropper; or a syringe.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “intraocular injection” refers to an injection that is administered by entering the eyeball of the patient.

The term “transzonular” refers to an injection administered through the ciliary zonule which is a series of fibers connecting the ciliary body and lens of the eye.

The term “intravitreal” refers to an injection administered through an eye of the patient, directly into the inner cavity of the eye.

The term “intraoperative” is defined as an action occurring or carried during, or in the course of, surgery.

The terms “anti-bacterial” and “antibiotic” used herein interchangeably, refer to substances or compounds that destroy bacteria and/or inhibit the growth thereof via any mechanism or route.

The term “anti-inflammatory” refers to substances or compounds that counteract or suppress inflammation via any mechanism or route.

The term “quinolone” for the purposes of this application refers to a genus of anti-bacterial compounds that are derivatives of benzopyridine and in some embodiments include fluorine atom, such as in the following structure (“fluoroquinolone”):

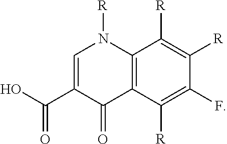

The term “corticosteroid” is defined as a compound belonging to a sub-genus of steroids that are derivatives of corticosterone, the latter having the chemical structure:

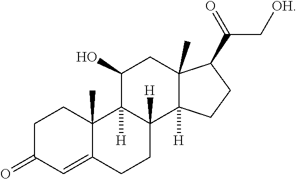

The term “salt” refers to an ionic compound which is a product of the neutralization reaction of an acid and a base.

The terms “solvate” and “hydrate” are used herein to indicate that a compound or a substance is physically or chemically associated with a solvent for “solvates” such as water (for “hydrates”).

The term “ether” refers to a chemical compound containing the structure R—O—R₁, where two organic fragments R and R₁ are connected via oxygen.

The term “ester” refers to a chemical compound containing the ester group R—O—C(O)—R₁, connecting two organic fragments R and R₁.

The terms “acetal” and “ketal” refer to a chemical compound containing the functional group R—C(R₁)(OR₂)₂, where R and R₂ are organic fragments and R₁ is hydrogen atom (for acetals), and is inclusive of “hemiacetals” where one R₂ (but not the other) is hydrogen atom; or where none of R, R₁ and R₂ is a hydrogen atom and each is an organic fragment (for ketals).

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

### B. Embodiments of the Invention

According to embodiments of the present invention, pharmaceutical compositions intended to prevent and/or treat inflammation and/or infections are provided. The compositions include an active component comprising, consisting essentially of, or consisting of a therapeutically effective quantity of an anti-bacterial agent (i.e., an antibiotic) and a therapeutically effective quantity of an anti-inflammatory agent (e.g., a corticosteroid). In some embodiments, the pharmaceutical compositions can be used for intraocular injections. In other embodiments the pharmaceutical compositions can be used for intra-articular or intra-lesional use. The compositions further include one or several pharmaceutically acceptable excipient(s) and one or several pharmaceutically acceptable carrier(s).

The concentration of the anti-bacterial agent in the pharmaceutical composition may be between about 0.01 mg/mL and about 50.0 mg/mL, such as between about 0.5 mg/mL and about 10 mg/mL, for example, about 1.0 mg/mL. The concentration of the anti-inflammatory agent in the pharmaceutical composition may be between about 0.1 mg/mL and about 100.0 mg/mL, such as between about 5.0 mg/mL and about 50.0 mg/mL, for example, about 15.0 mg/mL.

According to further embodiments, the anti-bacterial agent to be employed in the active component of the composition may be selected from the group of quinolones, including fluoroquinolones, and suitable derivatives of the same, such as pharmaceutically acceptable salts, hydrates or solvates thereof. In one embodiment, fluoroquinolone that may be so employed is moxifloxacin (chemically, 1-cyclopropyl-7-[(1S,6S)-2,8-diazabicyclo-[4.3.0]non-8-yl]-6-fluoro-8-methoxy-4-oxo-quinoline-3-carboxylic acid), which is available, e.g., under trade name Avelox® from Bayer Healthcare Corp. of Wayne, N.J., and under other trade names from other suppliers such as Alcon Corp. and Bristol-Myers Squibb Co. and has the following chemical structure:

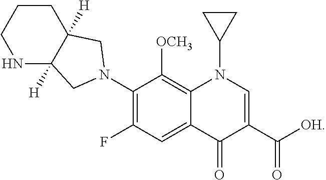 [SMILES: COc1c(N2C[C@@H]3CCCN[C@@H]3C2)c(F)cc2c(=O)c(C(=O)O)cn(C3CC3)c12]

A non-limiting example of a possible alternative fluoroquinolone antibiotic that may be used instead of, or in combination with, moxifloxacin is gatifloxacin. In some embodiments one or several glycopeptide antibiotic(s), or a combination of some or all of them, may be optionally used as a part of the anti-bacterial agent, in combination with moxifloxacin. One example of such an acceptable additional glycopeptide antibiotic is vancomycin which can be introduced into the pharmaceutical composition at a concentration between about 1 mg/mL and about 100.0 mg/mL, such as between about 5.0 mg/mL and about 50.0 mg/mL, for example, about 10.0 mg/mL. Vancomycin is available under the trade name Vancocin® from Eli Lilly & Co. of Indianapolis, Ind. Other acceptable additional glycopeptide antibiotics that may be used include teicoplanin, telavancin, decaplanin, ramoplanin, gentamicin, tobramycin, amikacin and cefuroxime.

According to further embodiments, the anti-inflammatory agent to be employed in the active component of the composition may be selected from the group of corticosteroids, such as derivatives of corticosterone, and pharmaceutically acceptable salts, hydrates, solvates, ethers, esters, acetals and ketals thereof. For example, a product obtained as a result of a chemically reasonable substitution of any hydrogen and/or hydroxyl group in the molecule of corticosterone may be used. In one embodiment, a corticosteroid that can be so utilized is triamcinolone (chemically, (11β,16α)-9-fluoro-11,16,17,21-tetrahydroxypregna-1,4-diene-3,20-dione) having the following chemical formula:

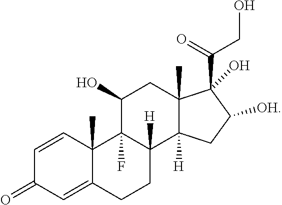

In another embodiment, a corticosteroid that can be so utilized is triamcinolone acetonide (chemically, (4aS,4bR,5S,6aS,6bS,9aR,10aS,10bS)-4b-fluoro-6b-glycoloyl-5-hydroxy-4a,6a,8,8-tetramethyl-4a,4b,5,6,6a,6b,9a,10,10a,10b,11,12-dodecahydro-2H-naphtho[2′,1′:4,5]indeno[1,2-d][1,3]dioxol-2-one) which is a ketal derivative of triamcinolone available, e.g., under the trade name Kenalog® from Bristol-Myers Squibb Co. of Princeton, N.J. and under other trade names from other suppliers, and having the following chemical formula:

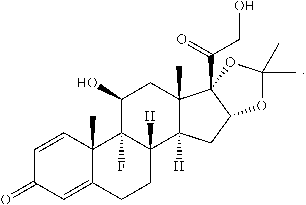

Other corticosteroids, or a combination of some or all of them, may be used instead of all or a portion of triamcinolone and/or of all or a portion of triamcinolone acetonide. Some non-limiting examples of such acceptable other corticosteroids include triamcinolone diacetate, triamcinolone benetonide, triamcinolone furetonide, triamcinolone hexacetonide, betamethasone acetate, dexamethasone, fluorometholone and fluocinolone acetonide.

As mentioned above, the pharmaceutical composition that is the subject matter of the instant application may further optionally include one or several pharmaceutically acceptable excipient(s). Those having ordinary skill in the art will be able to select the suitable excipient(s). It is worth mentioning that when moxifloxacin is used in pharmaceutical formulations, it is often difficult to obtain a stable suspension of another product (e.g., a corticosteroid such as triamcinolone acetonide) that is present in the same formulation and that needs to be in a form of a stable suspension. Without being bound by any particular scientific theory, such difficulties in obtaining the stable suspension are believed to be caused by moxifloxacin's tendency to deactivate many suspending agents resulting in unacceptable coagulation, clumping and flocculation. As a result, normal delivery through a typical 27-29 gage cannula is often difficult or even impossible.

Therefore, it is desirable to select an excipient that is stable in the presence of moxifloxacin and can, therefore, be used as a solubilizing and suspending agent to ensure that the corticosteroid such as triamcinolone acetonide safely forms a stable suspension even when moxifloxacin is also present in the same formulation. Numerous attempts by others to produce a stable moxifloxacin/triamcinolone acetonide pharmaceutical composition suitable for intraocular injection have not been successful.

In some embodiments, an excipient that can be used as a solubilizing and stabilizing agent to overcome the above-described difficulties and thus to obtain a stable suspension of the corticosteroid such as triamcinolone acetonide may be a non-ionic polyoxyethylene-polyoxypropylene block copolymer having the following general structure:

HO—(CH₂—CH₂—O)ₓ—(C₃H₆—O)_{y}—(CH₂—CH₂—O)ₓ—H,

wherein x is an integer having the value of at least 8 and y is an integer having the value of at least 38.

If a non-ionic polyoxyethylene-polyoxypropylene block copolymer is used as a solubilizing and stabilizing agent in the pharmaceutical compositions of the instant invention, its contents in the overall composition may be between about 0.01 mass % and about 10.0 mass % such as between about 1.0 mass % and about 8 mass %, for example, about 5.0 mass %.

One non-limiting example of a specific non-ionic polyoxyethylene-polyoxypropylene block copolymer that can be used as a solubilizing and stabilizing agent in the pharmaceutical compositions of the instant invention is the product known under the trade name Poloxamer 407® (poly(ethylene glycol)-block-poly(propylene glycol)-block-poly(ethylene glycol)) available from Sigma-Aldrich Corp. of St. Louis, Mo., with the molecular weight of the polyoxypropylene portion of about 4,000 Daltons, about a 70% polyoxyethylene content, the overall molecular weight of between about 9,840 Daltons and about 14,600 Daltons and having the following chemical structure:

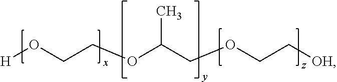

wherein each x and z is an integer having the value between about 78 and about 116, and y is an integer having the value of about 69.

Non-limiting examples of some other excipients and carriers that may be used in preparing in the pharmaceutical compositions of the instant invention include polysorbate (an emulsifier), edetate calcium disodium (EDTA, a chelating agent), hydrochloric acid (the pH adjuster) and sterile water.

According to further embodiments, methods for fabricating the above-described pharmaceutical compositions are provided. A one-batch formulation method may be used, where the components of the pharmaceutical formulation can be combined in single container; the components may be added to the container simultaneously or consecutively.

In one exemplary, non-limiting procedure, a quantity of an anti-bacterial agent such as moxifloxacin may be placed into a mixing container followed by adding a quantity of sterile water and hydrochloric acid to obtain a slightly acidic mixture (e.g., having pH of about 6.5) which is stirred until a clear solution is obtained. In case of the moxifloxacin/HCl system, the solution is stable, allowing the formulation to remain a closed system, thus preventing contamination and the loss of sterility.

Next, a quantity of corticosteroid such as micronized triamcinolone acetonide, a quantity of Poloxamer 407®, a quantity of edetate calcium disodium and a quantity of polysorbate 80 may be all added to be combined in the same container with the already prepared moxifloxacin/HCl solution and stirred together (e.g., by spinning) for a period of time, e.g., about 6 hours, until a homogenous suspension has been obtained. The resulting suspension may then be transferred into single dose vials, capped, sealed, autoclaved and shaken until cool. Finally, a complete testing for sterility and the presence of endotoxins may be performed on the product according to commonly used methods known to those having ordinary skill in the art.

Pharmaceutical compositions prepared as described above can be used to prevent complications that may arise after ophthalmic surgical operations and procedure. For example, the formulations can be used during any intraocular surgery, such as cataract surgery, planned vitrectomy or glaucoma procedures, to prevent or at least substantially reduce the risk of post-surgery complications, such as the development of endophthalmitis or cystoid macular edema (CME), without having the patient use pre- or post-operative topical ophthalmic drops. Individuals with evidence of endophthalmitis from prior surgical procedures or traumatic ocular penetration will benefit from concurrent injection of these formulations to sterilize infection and reduce damaging inflammation.

Pharmaceutical formulations described herein can be delivered via intraocular intravitreal injection which can be transzonular, or, if desired not transzonular. Intraocular intravitreal injection of this formulation, whether done via transzonular or via direct pars plana (trans-scleral) injection, delivers potent broad spectrum antibiotics directly into the suppurative tissue without requiring the urgent compounding of multiple individual medications or multiple individual injections into the eye.

Typically, a pharmaceutical composition described above will be intraocularly administered to a mammalian subject (e.g., humans, cats, dogs, other pets, domestic, wild or farm animals) in need of emergent, urgent or planned ophthalmic surgery treatment. The effect achieved by such use of pharmaceutical composition described above may last up to four weeks. The composition is to be injected intravitreally and trans-zonularly using methods and techniques known to those having ordinary skilled in the art of ophthalmology. In some embodiments, the injection can be intraoperative.

Typically, the delivery through a typical 27 gauge cannula can be employed utilizing a 1 mL TB syringe, with attention to re-suspending the formulation using momentary flicks and shakes just prior to injection. The medicinal volume (i.e., dosage) required of this formulation varies based on the type of intraocular procedure, the degree of postoperative inflammation induced or anticipated, the risk assessment for postoperative infection, and anatomic considerations regarding the available volume for the injection being added to a closed intraocular space.

It is worth mentioning that while intracameral (that is, anterior chamber) injections are within the scope of the instant invention such injections instead of posterior chamber (intravitreal) injection may not be satisfactory in some cases, as the suspension clogs the trabecular meshwork and aggravates intraocular drainage, resulting in an intraocular pressure rise postoperative. This is avoided with intravitreal injection, in addition to retaining the formulation components into the protein matrix of the vitreous for a greater duration. Anterior chamber wash out occurs over hours (antibiotic in solution) and days (steroid in suspension), while intravitreal injection is retained for weeks.

In alternative embodiments, if desired or necessary the formulations may also be delivered in the form of eye drops or eye sprays, as well as via subconjunctival injection, intraocular intracameral injection, sub-tenon injection, intra-articular injection or intra-lesional injection, particularly, in, but not limited to, some cases when necessary to deliver additional medication when local ocular inflammation and extra-ocular infection need suppression. Intravitreal delivery of steroids has historically been used to treat clinically significant cystoid macular edema (CME); the application of this formulation into the vitreous during routine intraocular procedures brings more aggressive prophylaxis against CME occurrence. Additionally, the suspension of this formulation is useful for staining vitreous during planned and unplanned vitrectomies, improving visualization of this otherwise transparent intraocular tissue, improving vitrectomy outcomes and reducing complications resulting from inadequate or tractional vitreous removal. In still further embodiments, there is also envisioned intra-canalicular delivery, i.e., delivery via a lacrimal canaliculus implant.

In some further alternative embodiments, instead of delivering the above-described compositions comprising both anti-bacterial and anti-inflammatory agents, consecutive injections may be used instead, if desired. For example, triamcinolone may be injected first, immediately followed by the injection of moxifloxacin or vice versa.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular ophthalmological condition being treated.

In additional embodiments, pharmaceutical kits are provided. The kit includes a sealed container for the storage of pharmaceutical compositions, e.g., without limitation a pharmaceutically suitable vial. The sealed container contains one of the above-described pharmaceutical compositions. The kit further includes at least one drug delivery device(s) that is (are) enclosed in the same packaging with the sealed container. An instruction for the use of the composition and the information about the composition are to be included in the kit.

The drug delivery device(s) included within the kit may be device(s) for delivery by injection, and/or for delivery via eye drops and/or for delivery via spray. If the drug delivery device(s) is (are) a device(s) for delivery by injection the kit includes, e.g. without limitation, at least one needle and/or at least one cannula suitable for ophthalmological application. The kit may optionally further comprise at least one syringe. Those having ordinary skill in the art will select the specific kind(s) of needle(s) and/or cannula(s) and, if desired, syringe(s) that are appropriate for the inclusion into the kit.

In other embodiments the kit may include the drug delivery device(s) for delivery via eye drops and may be, e.g. without limitation, at least one eyedropper, and/or at least one pipette. Those having ordinary skill in the art will select the specific kind(s) of eyedropper (s) and/or pipette(s) that are suitable.

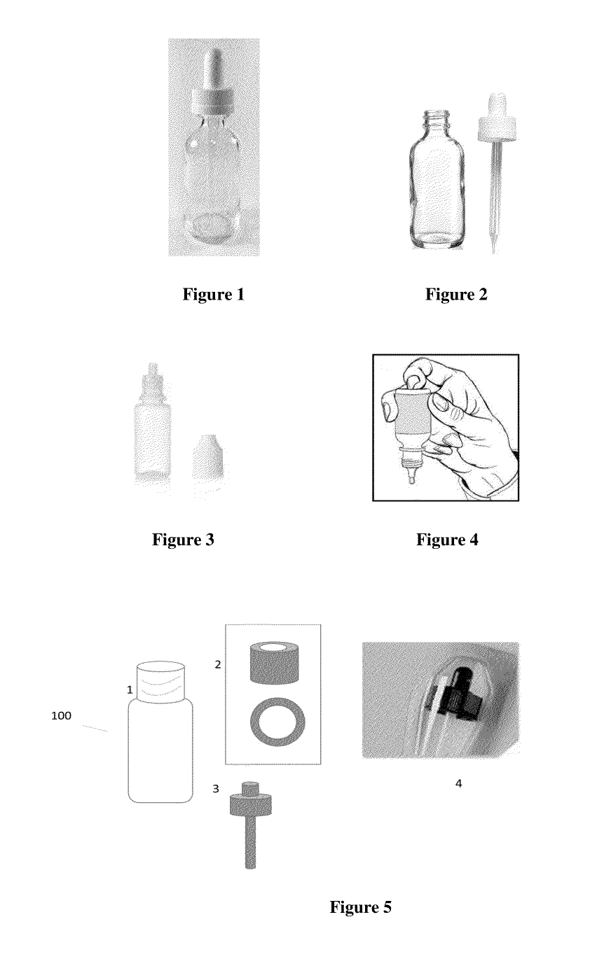

In yet other embodiments the kit may include a combination storage/administration drug delivery device(s) for delivery via eye drops. Such combination device(s) may include a sealed container that is reversibly convertible from a storage apparatus to a delivery device. The sealed container in these embodiments comprises a vial or a bottle containing the pharmaceutical composition and an eyedropper or pipette as the drug delivery device. The sealed container can be configured to receive the eyedropper so that the latter is detachably integrated with the vial. One such combination storage/administration device is shown on FIG. 1. In this combination device the sealed container serves as the storage apparatus for storing the pharmaceutical composition when the eyedropper is attached to the vial and serves as a stopper. When the pharmaceutical composition is to be administered the eyedropper is removed from the vial (as shown by FIG. 2) and is used for delivery via drops. Following the delivery the eyedropper is returned to the vial and the device is again to be used for storage of the pharmaceutical composition.

In some other embodiments the kit may include the drug delivery device(s) for delivery via eye drops wherein the sealed container is a squeezable vial comprising a tip (see FIG. 3). When the vial is squeezed (as shown by FIG. 4) the drops are formed at the tip thus allowing the delivery the pharmaceutical composition. Any suitable polymeric material can be used for fabricating the squeezable vial material, e.g., without limitations, poly(ethyleneterephthalate), poly(vinyl chloride), poly(ethylene), poly(propylene), etc. Those having ordinary skill in the art will select the appropriate polymer.

In further embodiments, the kit may include a convertible delivery device that combines features allowing administering the pharmaceutical composition via injection and via eye drops. This device includes a container that can be used for delivery via injection which container can be converted to enable delivery via eye drops, and vice versa. Several specific embodiments of such convertible delivery devices are illustrated schematically by FIGS. 5-7.

FIG. 5 shows a convertible delivery device 100 that includes a threaded plastic or glass vial or bottle 1, a needle-penetrable screw top 2 with injector compatible septa. Separately included into the kit is an eye drop dispenser or dropper 3, which is also shown separately in the package 4. To use the device, the screw top 2 may be removed opening the vial 1 followed by using the dropper 3 for collecting the pharmaceutical composition contained in the vial 1 and by dispensing the composition from the dropper 3. Alternatively, if desired, the screw top 2 may remain in place (i.e., being screwed on the vial 1) and the needle and/or cannula optionally included sealed container (not shown) can be used to puncture the screw top 2 to collect the composition followed by injecting it to the patient using a syringe which can be also optionally included.

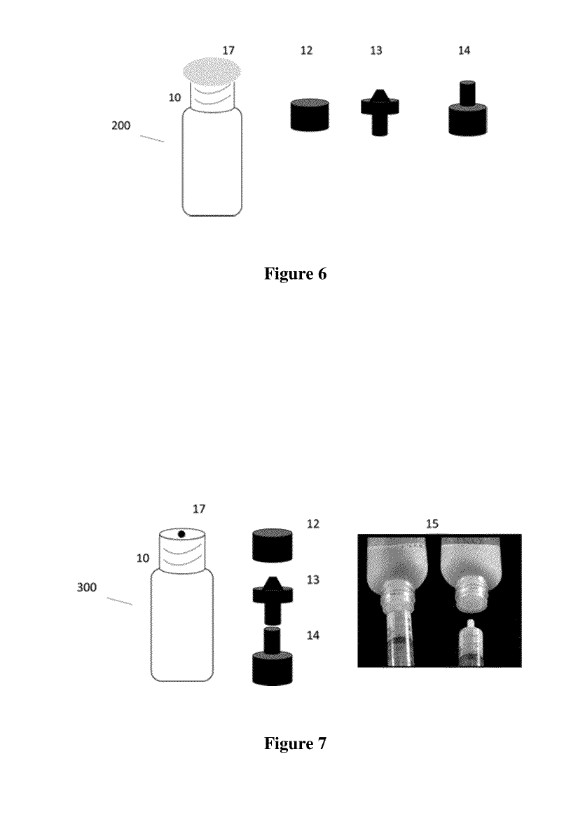

FIG. 6 shows a device 200 which includes a needle-impenetrable heat seal 17 on the opening of the squeezable container such as vial 10, a screw-on cap 12, a snap eye drop element 13 and a cap 14 for the snap eye drop element. The cap 14 can be manufactured by those having ordinary skill in the art so that it can be screwed on the vial 10. FIG. 6 shows the situation when the cap 12 was removed from the vial 1 revealing the heat seal 17. If it is desirable to apply the pharmaceutical composition as eye drops, the snap eye drop element 13 can be inserted inside the cap 14 followed by screwing up the assembly on the vial 10. When the assembly is so screwed on, the snap eye drop element 13 will break the seal 17 and the composition can be then delivered to the patient by squeezing the vial 10, i.e., in the manner illustrated by FIG. 4. Alternatively, the heat seal 17 can be removed and the needle and/or cannula optionally included into the kit (not shown) can be used to collect the composition followed by injecting it to the patient using a syringe which can be also optionally included.

A variation 300 of this device is shown in FIG. 7, but the seal 17 is in this case penetrable. The same procedure can be used for eye drop application as that described above where the device of FIG. 6 is discussed. Otherwise, the seal 17 can be penetrated using an injector (e.g., a needle) for delivery by injection. This step is illustrated by FIG. 7, item 15.

In yet additional embodiments, the sealed container included in the kit may be a syringe pre-filled with the measured quantity of the pharmaceutical composition to be administered (not shown by figures). The quantity in either weight or volume units can be printed on the outer surface of the syringe or otherwise indicated on the information sheet inserted into the kit. Such embodiments can provide an extra precise measurement of the amount of the medication to be delivered by injection. Since the quantity of the composition was already measured, the medical practitioner injecting the composition need not measure the quantity thereof, thus providing additional convenience and saving time for the practitioner. The pre-filled syringe kit may further optionally include a needle or a cannula for administering the pharmaceutical composition which can be either already attached to the syringe or be detached. In other embodiments, the kit does not include a needle or a cannula, in which case the medical practitioner may use a needle or a cannula obtained elsewhere.

The following examples are provided to further elucidate the advantages and features of the present invention, but are not intended to limit the scope of the invention. The examples are for the illustrative purposes only. USP pharmaceutical grade products were used in preparing the formulations described below.

### C. Examples

### Example 1

### Preparing a Pharmaceutical Composition

A pharmaceutical composition was prepared as described below. The following products were used in the amounts and concentrations specified:

(a) about 1.5 g of triamcinolone acetonide, at a concentration of about 15.0 mg/mL;

(b) about 0.1 g of moxifloxacin hydrochloride, at a concentration of about 1.0 mg/mL;

(c) about 1 mL of polysorbate 80, at a concentration of about 1.0 mass %;

(d) about 0.2 g of edetate calcium disodium, at a concentration of about 0.2 mass %;

(e) about 1 g of Poloxamer 407®, at a concentration of about 1.0 mass %;

(f) hydrochloric acid, to adjust pH to about 6.5; and

(g) about 100.0 mL of sterile water for injection.

Moxifloxacin hydrochloride was placed into a de-pyrogenated beaker with a spin bar. Sterile water for injection was added to about ⅓ of the volume of the beaker. While spinning, moxifloxacin was dissolved by adding hydrochloric acid until a clear solution having the final pH of about 6.5 was obtained.

The solution was combined with micronized triamcinolone acetonide, Poloxamer 407®, edetate calcium disoudium and polysorbate 80 and allowed to spin for about 6 hours until a hydrated and homogenous suspension was obtained.

The suspension was transferred into de-pyrogenated, single dose vials (2 mL size), capped and sealed, followed by autoclaving and shaking the vials until cool. Complete sterility and endotoxin testing was performed by an outside laboratory to ensure safety.

The formulation prepared as described above was tested for stability after 6 months of storage. After this period of storage no loss of potency was observed (as measured by HPLC); the formulation was visually stable at room temperature and readily re-suspended with gentle shaking with no increase of particle size or flocculation.

### Example 2

### Preparing a Pharmaceutical Composition Containing Vancomycin

A pharmaceutical composition was prepared as described in Example 1, supra. The composition was autoclaved and sonicated for about 60 minutes and about 96 mL of the composition were combined with about 4 mL of vancomycin at a concentration of about 250 mg/mL. The pH of the mixture was adjusted to about 6.0-6.5 using hydrochloric acid. The product was then transferred into vials (at about 1 mL plus 5 drops per vial) and frozen. The product has kept its stability and potency for at least six months.

### Example 3

### Using a Pharmaceutical Composition

A pharmaceutical composition fabricated as described in Example 1, supra, was administered to about 1,600 patients. To each, it was introduced using intravitreal transzonular injection. The injection was intraoperative. Only a very few patients, at the rate of about only 1 in 4,000, have developed any infection or suffered from other side effects that required further treatment, which is a substantial improvement over a typical rate of about 8% for the patients that did not receive the injection.

Although the invention has been described with reference to the above examples, it will be understood that modifications and variations are encompassed within the spirit and scope of the invention. Accordingly, the invention is limited only by the following claims.

What is claimed is:

1. A pharmaceutical kit, comprising:
(1) a sealed container containing an ophthalmic pharmaceutical composition comprising:
(a) a therapeutic component consisting essentially of:
(a1) a therapeutically effective quantity of an anti-bacterial agent independently selected from the group consisting of a quinolone, a fluorinated quinolone and a pharmaceutically acceptable salt, hydrate, solvate or N-oxide thereof; and
(a2) a therapeutically effective quantity of an anti-inflammatory agent independently selected from the group consisting of a corticosteroid and a pharmaceutically acceptable salt, hydrate, solvate, ether, ester, acetal and ketal thereof;
(b) optionally, at least one pharmaceutically acceptable excipient suitable for intraocular administration; and
(c) optionally, a pharmaceutically acceptable carrier therefor suitable for intraocular administration;
(2) one or several drug delivery devices for administering the pharmaceutical composition; and
(3) an instruction for the use of the composition.

2. The kit of claim 1, wherein the drug delivery device is selected from the group consisting of devices for delivery by injection, devices for delivery via eye drops and devices for delivery via spray.

3. The kit of claim 2, wherein the drug delivery device is a device for delivery by injection selected from the group consisting of a needle and a cannula.

4. The kit of claim 3, further comprising at least one syringe to be used in conjunction with the needle and/or the cannula.

5. The kit of claim 2, wherein the drug delivery device is for delivery via eye drops and is selected from the group consisting of an eyedropper and a pipette.

6. The kit of claim 1, wherein the sealed container is:
(a) a vial containing the pharmaceutical composition; and
(b) the drug delivery device is an eyedropper detachably integrated with the vial for administering the pharmaceutical composition,
wherein the vial serves as a storage apparatus for storing the pharmaceutical composition when the eyedropper is attached to the vial, and the eyedropper is used to administer the pharmaceutical composition when the eyedropper is detached from the vial.

7. The kit of claim 6, further configured to allow delivery by injection, wherein the seal of the vial is penetrable by an injector and the drug delivery device is an injector selected from the group consisting of a needle and a cannula.

8. The kit of claim 1, wherein the sealed container is a squeezable vial comprising a tip as the drug delivery device that can be used to deliver the pharmaceutical composition when the vial is squeezed.

9. The kit of claim 8, wherein the squeezable vial is fabricated of a polymeric material selected from the group consisting of poly(ethyleneterephthalate), poly(vinyl chloride), poly(ethylene) and poly(propylene).

10. The kit of claim 1, wherein the sealed container is a threaded plastic or glass container having a needle-penetrable and/or a cannula-penetrable removable stopper screwed on the container using the thread, the kit further comprising:
(a) an eye drop dispenser as the drug delivery device;
(b) optionally, a needle or a cannula; and
(c) optionally, a syringe.

11. The kit of claim 1, wherein the sealed container is a squeezable vial having a removable needle-impenetrable or cannula-impenetrable heat seal attached to the opening of the vial, the kit further comprising:
(a) a snap eye drop element as the drug delivery device that is attachable to the squeezable vial and is capable of breaking the heat seal when attached to the vial for administering the pharmaceutical composition via drops;
(b) optionally, a needle or a cannula; and
(c) optionally, a syringe.

12. The kit of claim 1, wherein the sealed container is a squeezable vial having a needle-penetrable or cannula-penetrable seal attached to the opening of the vial, the kit further comprising:
(a) a snap eye drop element as the drug delivery device that is attachable to the squeezable viable and is capable of breaking the seal when attached to the vial for administering the pharmaceutical composition via drops;
(b) optionally, a needle or a cannula; and
(c) optionally, a syringe.

13. The kit of claim 1, wherein the sealed container is a syringe pre-filled with the measured quantity of the pharmaceutical composition, and the drug delivery device is a needle or a cannula for administering the pharmaceutical composition.

14. The kit of claim 1, wherein the anti-bacterial agent is a fluorinated quinolone.

15. The kit of claim 14, wherein the fluorinated quinolone is selected from the group consisting of moxifloxacin and gatifloxacin.

16. The kit of claim 14, wherein the fluorinated quinolone is moxifloxacin.

17. The kit of claim 1, wherein the corticosteroid is selected from the group consisting of triamcinolone, triamcinolone acetonide, triamcinolone diacetate, triamcinolone benetonide, triamcinolone furetonide, triamcinolone hexacetonide, betamethasone acetate, dexamethasone, fluorometholone, fluocinolone acetonide, prednisone, prednisolone, methylprednisone, corticol, cortisone, fluorocortisone, deoxycorticosterone acetate, aldosterone, budesonide and derivatives, analogs or combinations thereof.

18. The kit of claim 17, wherein the corticosteroid is selected from the group consisting of triamcinolone, dexamethasone, prednisone and prednisolone.

19. The kit of claim 17, wherein the corticosteroid is triamcinolone.
